(19) United States
(12) Patent Application Publication
(10) Pub. No.: US 2024/0260886 A1
(43) Pub. Date: Aug. 8, 2024

(54) ESOPHAGUS PH-MYOELECTRICITY COMBINED ELECTRODE CATHETER

(71) Applicant: THE FIRST AFFILIATED HOSPITAL OF GUANGZHOU MEDICAL UNIVERSITY (GUANGZHOU RESPIRATORY CENTER, Guangzhou, Guangdong (CN)

(72) Inventors: Yuanming LUO, Guangzhou, Guangdong (CN); Shanfeng LIANG, Guangzhou, Guangdong (CN)

(21) Appl. No.: 18/562,727

(22) PCT Filed: Dec. 15, 2020
(86) PCT No.: PCT/CN2020/136448

§ 371 Date: Nov. 20, 2023

(30) Foreign Application Priority Data
May 19, 2020 (CN) 202010424512.6

(51) Int. Cl. A61B 5/392; A61B 5/00

(52) U.S. Cl. CPC A61B 5/392; A61B 5/4211

(57) ABSTRACT

An esophagus PH-myoelectricity combined electrode catheter, comprising a catheter body (9), wires for PH electrodes (11) and wires for diaphragm EMG (12); two PH electrodes (1, 2), multipair diaphragm EMG electrodes (3, 4, 5, 6, 7) and a reference electrode (8) are attached on the surface of the catheter body (9); the two PH electrodes (1, 2) are respectively located at the distal end and the middle part of the catheter (9), multipair diaphragm EMG recording electrodes (3, 4, 5, 6, 7) are located between the two PH electrodes (1, 2), and the reference electrode for diaphragm EMG recording (8) is located at the proximal end of the PH electrode (2) at the middle part; both the PH electrodes (1, 2) are connected to the PH wire (11) inside the catheter (9), PH signal extension wire (11) is further connected to PH processor joint (13); the reference electrode (8) and the electrodes for recording diaphragm EMG (3, 4, 5, 6, 7) are connected to the wires (12) inside the catheter (9), and the diaphragm EMG signal extension wires (12) are connected to socket for bio-amplifier (14). The esophagus PH-myoelectricity combined electrode catheter can easily and accurately position PH electrodes to stomach and esophagus to reduce discomfort. With this technique gastroesophageal reflux and diaphragm function could be assessed and the potential cause of gastroesophageal reflux could be determined.

### FIELD OF THE INVENTION

The present disclosure relates to medical devices, and more particularly, to an esophagus PH-myoelectricity combined electrode catheter.

### BACKGROUND OF THE INVENTION

Gastroesophageal reflux is mainly acidic reflux, which can be manifested as acid reflux, heartburn, retrosternal pain, chronic cough, etc. The main causes for gastroesophageal reflux include dysfunction of lower esophageal sphincter, weakened contraction of the diaphragmatic crura, and decreased esophageal peristalsis. Theoretically, diaphragm disfunction may lead to weakened contraction of the diaphragmatic crura and reduced pressure of lower esophageal sphincter, thus resulting in gastroesophageal reflux. However, at present, there is no single catheter that enables simultaneously accurate detection of diaphragm functions and diagnosis of gastroesophageal reflux in clinical practice.

24-hour esophagus PH monitoring is a method for detecting the presence of gastroesophageal reflux. The accurate placement of an esophagus PH electrode catheter is critical for correct diagnosis. It is desirable that a proximal PH electrode be placed 5 cm away from a proximal edge of the lower esophageal sphincter. The current methods for catheter placement in clinical practice are based on manometry and pH gradient along the esophagus.

The esophagus manometry is based on a high-pressure zone created by the the lower esophageal sphincter. The high-pressure zone is a major barrier for gastroesophageal reflux. The manometry usually requires placement of two catheters, increasing the discomfort of the patient. Moreover, the equipment required for manometry is relatively complex and costly. In addition, most patients with gastroesophageal reflux the high-pressure zones of lower esophageal sphincter has been destructed, leading to difficult in accurate placement of the PH electrode catheter.

In the pH gradient method, electrodes are placed based on the differences in pH values between the stomach and the esophagus. However, in patients with hyperchlorhydria or hypochlorhydria, the PH gradients may disappear along the esophagus and the stomach, which affects localization.

The esophagus manometry and the PH gradient method described above are subject to changes in many factors, which interfere correct placement of the catheter leading to difficulty in judgment of gastroesophageal reflux and diaphragm functions.

### SUMMARY OF THE INVENTION

### Technical Problems

The aims of the invention are to overcome the above disadvantages in placement of catheter to provide an esophagus PH-myoelectricity combined electrode catheter for detection of gastroesophageal reflux and diaphragm function simultaneously. The catheter can be easily located to the proper place for detection of diaphragm functions, which is useful to discover the potential cause of gastroesophageal reflux.

### Technical Solutions

The object of the present disclosure is achieved by the following technical solution. The esophagus PH-myoelectricity combined electrode catheter includes a catheter body, the PH sensor wires, and the diaphragm electrode wires. There are two PH sensors, a multipair diaphragm EMG electrodes, and a reference electrode which are attached on the surface of the catheter body. The two PH sensors are arranged at the distal end and the middle part of the catheter while the multipair diaphragm EMG recording electrodes are all located between the two PH sensors. The reference electrode is located at the proximal part of the catheter near to the proximal PH sensor. The two PH sensors are separately connected to two different PH wires inside the catheter. The signal output detected by PH sensors is passed to PH processor through the PH wires. The reference electrode and the multipair diaphragm EMG recording electrodes are all connected to the wires inside the catheter and are further connected to a bio-amplifier.

Preferably, there are five electrodes for recording diaphragm EMG with an equal distance between two adjacent electrodes. The gap between the distal PH sensor and the most distal electrode for recording diaphragm EMG is greater than 5 cm while the gap is equal to 5 cm between the proximal PH sensor and the electrode for recording diaphragm EMG located at the middle position.

Preferably, the distance between two adjacent electrodes for recording diaphragm EMG is 1 cm.

Preferably, the five electrodes for recording diaphragm EMG form three pairs of electrodes which are the same in terms of distance between electrodes within a pair. There is one electrode which is a common electrode for two pairs of electrodes.

Preferably, the electrodes for recording diaphragm EMG including the reference electrode are all 1 cm in length.

Preferably, the two PH sensors are both less than 1 cm in length.

Preferably, the distal end of the catheter is sealed with a cap and the proximal end of the catheter have connectors which connect wires for PH sensors and the electrodes for recording multiopair diaphragm EMG respectively.

### Beneficial Effects

Compared with the prior work, the present disclosure has the following advantages.

1. The esophagus PH-myoelectricity combined catheter integrates the electrodes for recording diaphragm EMG and PH sensors which can simultaneously detect the diaphragm EMG and the PH value in the stomach and in the esophagus. The catheter can not only be accurately placed at the lower esophageal sphincter to adjudge whether there is gastroesophageal reflux, but also accurately record the diaphragm myoelectricity, which is helpful to disclose the cause of gastroesophageal reflux.

2. The esophagus PH-myoelectricity combined catheter does not require placement of two catheters which is needed in traditional way for diagnosis of gastroesophageal reflux, thereby simplifying the procedure for placement of the catheter, reducing the examination cost, and reducing the discomfort of patients.

### BRIEF DESCRIPTION OF THE DRAWINGS

FIG. 1 is a structural diagram of an esophagus PH-myoelectricity combined electrode catheter of the present disclosure.

In the drawing, 1 and 2 are PH sensors located at the end and the middle of the catheter, respectively. 3-7 are electrodes for diaphragm EMG recording. 8 is a reference electrode connecting to the earth. 9 is a catheter body, 10 is a bifurcated head, 11 PH sensor wires extension, 12 extension wires for recording diaphragm EMG, 13 is a PH processor connector, 14 bio-amplifier connector, 15 sealed cap, and I, II, and III are three pairs of electrodes for recording diaphragm EMG.

### DETAILED DESCRIPTION OF THE EMBODIMENTS

The present disclosure is described in further detail below with reference to the accompanying drawings and embodiments.

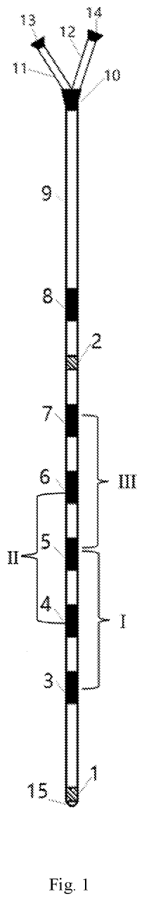

As shown in FIG. 1, the esophagus PH-myoelectricity combined electrode catheter includes a catheter body, two PH wires which connect to two electrodes for measurement of PH, and 6 diaphragm wires which connect to six electrodes respectively, for recording diaphragm EMG. The two PH sensors are separately arranged at the distal end and the middle part of the catheter. The electrodes for recording diaphragm EMG are all located between the two PH sensors while the reference electrode is located between the proximal PH sensor and the proximal end of the catheter. The PH sensors are both connected to the PH wire inside the catheter and is further connecting to the PH processor connector. The reference electrode and the diaphragm EMG electrodes are all connected to the wires inside the catheter, which are further connecting to a bio-amplifier connector. In practical, the PH processor connector and the bio-amplifier connect to a computer to process and display relevant signals. The reference electrode connects to the earth for better EMG signal quality.

Both PH sensors (PH 1 and PH 2) are 0.5 cm in length. The PH sensor 1 is close to the sealed cap at the distal end of the catheter. The PH sensor 1 is designed to detect the PH value in the stomach. When the catheter is in correct position for detection of gastroesophageal reflux, the PH 2 is placed 5 cm away from a proximal edge of the lower esophageal sphincter.

There are five diaphragm EMG electrodes which have an equal distance between two adjacent electrodes. The distance between PH 1 and the distal electrode for EMG recording is larger than 5 cm while the electrode 5 for EMG recording and the PH 2 sensor is 5 cm. The electrodes 3-7 and the reference electrode 8 for recording diaphragm EMG are all 1 cm in length. The distance between two adjacent electrodes for recording diaphragm EMG is 1 cm in length.

The five electrodes for recording diaphragm EMG form three pairs of electrodes which are exact the same in terms of the distance between electrodes within a pair. One electrode (electrode 5) is the common electrode for pair I (electrodes 1 and 5) and pair III (electrode 5 and 7). To position catheter to the gastric-esophagus properly, operator observe diaphragm EMG signals simultaneously recorded from three pairs of electrodes (pairs I, II and III). When pair I and pair III record signal with a similar amplitude but much larger than that recorded by the lead II, indicating that the electrode 5 for recording diaphragm EMG is at the level of esophageal sphincter. Consequently, the PH sensor 2 is at proximal 5 cm from the esophageal sphincter. Under the placement, the pair II record small diaphragm EMG because electrical potentials detected by electrodes 4 and 6 are almost equal and are cancellated. By measuring and analyzing the diaphragm EMG recorded from the esophagus PH-myoelectricity combined electrode catheter, the diaphragm functions can be assessed, which is helpful to find the potential cause of gastroesophageal reflux.

The distal end of the catheter is sealed with a cap, the proximal end of the catheter is designed to have a bifurcated head for connecting to PH processor and a bio-amplifier

The specific embodiments described above are preferred embodiments of the present disclosure, without limiting the present disclosure. Any other variations or other equivalent substitutes made without departing from the technical solution of the present disclosure shall be included in the protection scope of the present disclosure.

1. An esophagus PH-myoelectricity combined electrode catheter, comprising a catheter body, two wires inside the catheter connecting to PH sensors, and five wires inside the catheter connecting to electrodes for recording the diaphragm EMG. PH sensors and the electrodes for diaphragm EMG are attached on the surface of the catheter. The two PH sensors are located at the distal end and the middle part of the catheter, respectively. The five electrodes for EMG recording are located between the two PH sensors while the reference electrode is located between the proximal PH sensor and the proximal end of the catheter. The wires inside the catheter connect to PH sensors are further connected to a PH processor connector at the proximal end while the wires inside the catheter connect to the electrodes for recording diaphragm EMG are all separately connected to a bio-amplifier.

2. The esophagus PH-myoelectricity combined electrode catheter of claim 1, wherein there are five electrodes for recording diaphragm EMG which are 1 cm in length for each electrode and the distance between adjacent electrodes is 1 cm. The distal electrode for recording diaphragm EMG is 5 cm away from the distal PH sensor, while a space is also 5 cm between the proximal PH sensor and the middle electrode for recording diaphragm EMG

3. The esophagus PH-myoelectricity combined electrode catheter of claim 2, wherein the distance between two adjacent diaphragm electromyographic signal recording electrodes is 1 cm.

4. The esophagus PH-myoelectricity combined electrode catheter of claim 2, wherein the five electrodes for recording diaphragm EMG form three pairs of electrodes with the exact same distance between electrodes within a pair. The middle electrode is the common electrode for upper and lower pairs.

5. The esophagus PH-myoelectricity combined electrode catheter of claim 1, wherein the electrodes for recording diaphragm EMG and the reference electrode all are 1 cm in length.

6. The esophagus PH-myoelectricity combined electrode catheter of claim 1, wherein the two PH sensors are both less than 1 cm in length.

7. The esophagus PH-myoelectricity combined electrode catheter of claim 1, wherein the distal end of the catheter is sealed with a cap, the proximal end of the catheter has a bifurcated head which is further extended to PH processor connector for measurement of PH and extended to bio-amplifier connector.
